(19) United States
(12) Patent Application Publication
(10) Pub. No.: US 2019/0185896 A1
(43) Pub. Date: Jun. 20, 2019

(54) Stimulation of Regenerative Processes and Factors by Noble Gases

(71) Applicant: NOBILIS THERAPEUTICS, INC., Portland, OR (US)

(72) Inventors: Daqing Ma, London (GB); Hailin Zhao, London (GB); Kaizhi Lu, Chongqing (CN); Jiaolin Ning, Chongqing (CN); Jianteng Gu, Chongqing (CN); Thomas Ichim, San Diego, CA (US); Vlad Bogin, Portland, OR (US)

(73) Assignee: NOBILIS THERAPEUTICS, INC., Portland, OR (US)

(21) Appl. No.: 15/847,793

(22) Filed: Dec. 19, 2017

(51) Int. Cl. C12P 21/00; C07K 14/52; C07K 14/475; C07K 14/515

(52) U.S. Cl. CPC C12P 21/00; C07K 14/515; C07K 14/475; C07K 14/52

(57) ABSTRACT

The invention provides compositions, treatment means and protocols for induction of regenerative processes by administration of noble gases. In one particular embodiment the invention provides the stimulation of production of factors associated with augmentation of hematopoiesis, angiogenesis, and wound healing by exposure of cells, organs, or mammals to a noble gas. In a particular embodiment the invention provides the administration of argon as a noble gas capable of upregulating production of VEGF and angiopoietin.

### FIELD OF THE INVENTION

The present technology is directed to the filed inducing regenerative processes by the administration of noble gases and mixtures thereof.

### BACKGROUND OF THE INVENTION

Wound healing is the process responsible for the restoration of normal anatomical structures and functions after their disruption by an injury. Although the process re-establishes the role and morphology of the skin barrier, the maximum tensile strength of the scar is only approximately 70-80% of that of the original uninjured tissue [1]. What's more, the mechanism involved is complex and requires cross-talk between different cell types. Although overlapping in time, the process can be divided into four phases in sequential order in vivo, namely haemostasis, inflammation, proliferation and remodeling [1]. Wounds are categorised as either acute or chronic according to the duration of the healing process. Chronic wounds are the consequence of impaired wound healing, in which the repair process does not occur in order.

Angiogenesis is the formation of new blood vessels from pre-existing vasculature [2] and it happens almost throughout the repair process. The purpose of this is to establish a new vascular network to perfuse the hypoxic wound environment triggered by both initial microvessel disruptions from the insult and escalated cellular metabolic activities; hence an increased demand for oxygen and nutrients as repair proceeds. Angiogenesis entails interaction between cells and soluble mediators as well as immobilised ligands on the provisional ECM; it can also be driven by the sheer force of blood flow. Endothelial cells (ECs) are the major players of neovascularisation which migrate into the wound by three distinct mechanisms acting synergistically, namely chemotaxis, haptotaxis and mechanotaxis [2].

Wound healing is of major clinical importance for the successful management of conditions with chronic skin defects, such as burns injury and diabetes. At present, treatments to promote effective healing are limited; hence exploration of novel therapeutic strategies is needed. To date, chronic wounds have been observed in a variety of disease conditions; for instance in diabetic patients in the form of diabetic foot ulcers (DFUs). The pathogenesis of DFUs are influenced by both intrinsic and extrinsic factors [3]. Vasculopathy is one intrinsic factor identified in diabetes—although the luminal diameter of the microvasculature remains constant and there are no occluded vessels in these patients, abnormal blood flow, hence tissue ischaemia results in their lower extremities. Different approaches have been implemented or are under clinical investigation to enhance healing of DFUs. These include wound debridement, dressings, hyperbaric oxygen therapy, compression or negative pressure therapy, application of stem cells as well as skin grafts and substitutes[4]. However, as no ideal treatment exists for the condition, better alternatives are always being sought.

### SUMMARY OF THE INVENTION

Various aspects of the invention provided herein are enumerated in the following paragraphs:

Aspect 1. A method of inducing factors that stimulate hemangiogenic cells, said method comprising of contacting a noble gas with a mammalian tissue at sufficient concentration and duration to stimulate production of said factor.

Aspect 2. The method of claim 1, wherein, said hemangiogenic cell is a cell capable of hematopoiesis.

Aspect 3. The method of claim 2, wherein said cell capable of hematopoiesis is a cell expressing CD34.

Aspect 4. The method of claim 2, wherein said cell capable of hematopoiesis is a cell expressing CD133.

Aspect 5. The method of claim 2, wherein said cell capable of hematopoiesis is a cell expressing which possesses ability to reconstitute multilineage blood production in an animal in which endogenous hematopoietic cells have been ablated.

Aspect 6. The method of claim 2, wherein said multilineage blood production comprises of generation of de novo: a) platelets; b) erythrocytes; and c) leukocytes.

Aspect 7. The method of claim 1, wherein said hemangioblast cells is an endothelial progenitor cell.

Aspect 8. The method of claim 7, wherein said endothelial progenitor cell is capable of generating endothelial cells in tissue culture.

Aspect 9. The method of claim 7, wherein said endothelial progenitor cell expresses a marker selected from a group comprised of: a) CD133; b) CD34; and c) KDR-1.

Aspect 10. The method of claim 1, wherein said hemangioblast cell is an endothelial cell.

Aspect 11. The method of claim 10, wherein said endothelial cell expresses CD31.

Aspect 12. The method of claim 1, wherein said hemangioblast cell is a progenitor cell capable of generating progeny of either or both of the hematopoietic or endothelial lineage.

Aspect 13. The method of claim 1, wherein said noble gas is selected from a group comprising of: a) helium; b) argon; c) neon; d) krypton; and e) xenon.

Aspect 14. The method of claim 1, wherein said noble gas is argon.

Aspect 15. The method of claim 1, wherein said mammalian tissue contacted with said noble gas comprises of endothelial cells.

Aspect 16. The method of claim 1, wherein said mammalian tissue contacted with said noble comprises of myeloid lineage cells.

Aspect 17. The method of claim 1, wherein said stimulation of hemangiogenic cells is induction of proliferation.

Aspect 18. The method of claim 1, wherein said stimulation of hemangiogenic cells is induction of cytokine production.

Aspect 19. The method of claim 1, wherein said stimulation of hemangiogenic cells is induction of receptors associated with chemotaxis.

Aspect 20. The method of claim 1, wherein said factor capable of stimulating hemangiogenic cells is VEGF.

Aspect 21. The method of claim 1, wherein said factor capable of stimulating hemangiogenic cells is angiopoietin.

Aspect 22. A method of inducing production of VEGF in a fibroblast comprising of contacting said fibroblast with a noble gas for a sufficient concentration and exposure time to induce production of said VEGF.

Aspect 23. The method of claim 22, wherein said noble gas is argon.

Aspect 24. The method of claim 23, wherein said noble gas is argon at a concentration of 75% argon with 25% oxygen.

Aspect 25. A method of stimulating production of an angiogenic factor in an injured tissue comprising administering a therapeutically sufficient concentration of a noble gas to said injured tissue.

Aspect 26. The method of claim 25, wherein said noble gas is argon.

Aspect 27. The method of claim 26, wherein said argon is administered at a concentration of 75% argon and 25% oxygen.

Aspect 28. The method of claim 25, wherein said angiogenic factor is angiopoietin.

Aspect 29. The method of claim 25, wherein said angiogenic factor is VEGF.

Aspect 30. A method of stimulating migration of regenerative cells in an injured tissue comprising administering a therapeutically sufficient concentration of a noble gas to said injured tissue.

Aspect 31. The method of claim 30, wherein said noble gas is argon.

Aspect 32. The method of claim 31, wherein said argon is administered at a concentration of 75% argon and 25% oxygen.

Aspect 33. The method of claim 30, wherein said regenerative cells are endothelial progenitor cells.

Aspect 34. The method of claim 30, wherein said regenerative cells are hematopoietic stem cells or progenitor cells.

Aspect 35. A method for accelerating healing in an injured tissue comprising administering a therapeutically sufficient concentration of a noble gas to said injured tissue.

Aspect 36. The method of claim 35, wherein said noble gas is argon.

Aspect 37. The method of claim 35, wherein said argon is administered at a concentration of 75% argon and 25% oxygen.

### BRIEF DESCRIPTION OF THE DRAWINGS

The accompanying drawings, which illustrate one or more exemplary embodiments:

FIG. 1A shows stains establishing exposure of argon enhanced VEGF expression in vitro.

FIG. 1B shows a graph establishing exposure of argon enhanced VEGF expression in vitro.

FIG. 1C shows stains establishing exposure of argon enhanced a-SMA-expression in vitro.

FIG. 1D shows a graph establishing exposure of argon enhanced a-SMA-expression in vitro.

FIG. 2A shows stains establishing exposure of argon enhanced Angiopoietin 1 expression in vivo.

FIG. 2B shows a graph establishing exposure of argon enhanced Angiopoietin 1 expression in vivo.

FIG. 2C shows stains establishing exposure of argon enhanced VEGF expression in vivo.

FIG. 2D shows a graph establishing exposure of argon enhanced VEGF expression in vivo.

FIG. 3A shows stains establishing exposure of argon enhanced CD34 expression in vivo.

FIG. 3B shows a graph establishing exposure of argon enhanced CD34 expression in vivo.

FIG. 3C shows stains establishing exposure of argon enhanced TGF-β expression in vivo.

FIG. 3D shows a graph establishing exposure of argon enhanced TGF-β expression in vivo.

FIG. 4 are photos showing wound healing in mice was accelerated by treatment with Argon.

### DESCRIPTION

Despite the complex pathogenesis of diabetes, with various interdependent factors, it is desirable to develop novel strategies promoting angiogenesis to enhance blood supply to the affected areas, with the hope of restoring normal wound healing in conjunction with other technological advancements.

Use of noble gases in clinical settings has become increasingly popular during the past decade. Gases like xenon, helium and most recently argon have become targets of interest especially in the field of neural injury. Previously, our group published some encouraging results showing argon gas at a 75% concentration potently enhanced viability and proliferation of cerebral cortical neurons in vitro, regardless of presence or absence of OGD injury [5]. The cumulative evidence from both in vitro and in vivo investigation was promising. In addition, since argon exists in high abundance, as well as its non-narcotic nature when administered at normobaric pressure [6], it is undoubtedly a potential candidate to substitute xenon and to be explored in promoting survival and proliferation of other cell types such as ECs. If this is true, this would mean that in turn argon might possibly enhance angiogenesis, which is the main focus of this study in the context of wound healing. The aim of this invention is to use f argon exposure to accelerate wound healing, and to cover the molecular pathways underlying any beneficial effects of argon. The invention provides means of stimulating production of growth factors through treatment of various cells with noble gases.

In one embodiment of the invention, treatment with argon is used to stimulate production of growth factors such as VEGF, which stimulate angiogenesis and hematopoiesis. The invention provides means of accelerating wound healing, which is provided in the examples.

### Examples

### HUVEC Culture

HUVECs obtained from Lonza Ltd., UK were seeded in 75 cm² flasks previously treated with 10% gelatin (Sigma-Aldrich, UK) and cultured in EGM at 37° C., in a humidified 5% carbon dioxide (CO₂) aerobic incubator (Automatic CO₂ incubator, NuAire, Caerphilly, UK).

### Argon Exposure

Cells were kept in purposed-built airtight chambers with inbuilt valves and electric fan for gas exposure. Chambers were first flushed with their corresponding gas mixtures for 10 minutes to ensure the interior was only filled with gases of the desired composition. After that, rubber tubes on both sides of the chamber allowing the delivery and escape of gases were clamped to establish a closed system. The two chambers containing argon- and nitrogen-exposed cells were then kept in the incubator at 37° C. for 2 hours, along with the normoxia groups (NC and PC), which were not contained by the chambers. At the end of treatment, groups from the chambers were returned to normoxic environment within the incubator until subsequent collection. All gases were supplied by BOC Gases (Surrey, UK).

### Endothelial Tube Formation Assay (In Vitro Angiogenesis)

The assay was performed according to Invitrogen's “Endothelial Tube Formation Assay (In Vitro Angiogenesis)” protocol to determine the extent of argon in promoting HUVEC differentiation. In preparation, wells of 12-well plates were incubated with 50 μL Geltrex™ per cm² at 37° C. for 30 minutes. After the gel solidified, HUVECs in complete and incomplete LSGS medium were seeded at the density of 3.5-4.5×104 cells per 200 μL per cm² into the wells.

Straight after this, the cells underwent gas exposures (normal air for both PC and NC, 75% argon or 75% nitrogen) for 2 hours at 37° C. A PC group was included to show that tube formation occurred when stimulated by bFGF (contained in the complete LSGS medium only), which initiates angiogenesis in vivo.

At 0, 2 and 4 hours post-treatment, wells were examined under an inverted light microscope (Olympus, UK) at 100× magnification and imaged with a camera (Olympus c-5050, UK) down the eyepiece (30 random views per well). Once assessment was completed, plates were returned to resume incubation. Tube formation images were analysed by ImageJ analysis software.

### Wound of the Mice Skin

To create the excisional wounds, adult mice were anesthetized and the dorsal skin was shaved, after sanitizing with 70% ethanol, 4 full-thickness excisional wounds were generated with a 4-mm sterile punch (stiefel laboratories, Research Triangel Park, N.C.). On the dorsal skin using 4-mm-diameter dermal biopsy punches through the two layers of skin (Miltex Inc.).

### Immunohistochemistry

The perfusion fixed skin tissue was cryosectioned (thickness 30 μm) and mounted on Superfrost Plus glass slides for the process of immunostaining analysis. The sections were initially incubated by a solution of 0.3% hydrogen peroxide in methanol to quench endogenous peroxidase before being exposed to a blocking solution of 0.1M phosphate buffered saline (PBS) containing 0.3% Triton X-100 and 3% natural sheep serum (NSS). Following this process of blocking the sections were incubated overnight at 4% with rat polyclonal angiopoietin 1, VEGF, CD34, TGF-β (Abcam, UK). All sections were then washed in PBS with 0.3% Triton X-100 (PBST) before being incubated with a biotin-conjugated anti-rat secondary antibodies (1:200) (Millipore, UK) at room temperature for 2 hours. The sections were then washed with PBS before treatment with avidin-biotinperoxidase complex (ABC) solution (Vector Laboratories Ltd, California, USA) at room temperature for 45 minutes. After being washed with PBST sections were then exposed to nickel enhanced diaminobenzidine (DAB kit) to detect activity of peroxidase. Internal controls were used and received an incubation of the aforementioned blocking solution rather that a primary antibody; these sections showed no immunostaining. After dehydration of the sections with increasing concentrations of ethanol (50%, 70%, 90%, 100%) and clearing of boundaries using xylene, the slides were mounted using glass coverslips and DPX mounting medium (Lamb, Eastbourne, UK). For each animal photomicrographs of the stained sections were attained using a digital camera attached to a Zeiss Axiovert 200M inverted epifluorescence microscope (Zeiss, Jena, Germany). Four sections from each treatment group were examined under low and medium magnification to assess the intensity of the staining.

For in vitro fluorescence staining, cells were fixed in paraformaldehyde in 0.1 mol/l PBS solution. Cells were then incubated in 10% normal donkey serum in 0.1 mol/l PBS-Tween 20 and then incubated overnight with rabbit anti-angiopoietin-1 (1:200, Abcam), rabbit anti-TGF-β (1:200, Abcam), rabbit α-SMA (1:200, Abcam), followed by incubation with secondary antibody for 1 h. The slides were counterstained with nuclear dye DAPI and mounted with VECTASHIELD Mounting Medium (Vector Laboratories, Burlingame, Calif.). Sections were examined using an Olympus (Watford, UK) BX4 microscope.

### Cell Migration—Scratch Assay

The following was adopted from a previous published protocol (Liang et al., 2007) to study the influence of argon on migration of HUVECs. Prior to assay, HUVECs in EGM were re-suspended in incomplete LSGS medium and seeded onto pre-coated 12-well plates at a density of 2-3×105/well. The plates were incubated at 37° C. with 5% CO₂ overnight for the cells to adhere to the gelatin surface as well as to create a confluent monolayer. Use of low serum condition is crucial as it ensures survival and attachment of cells while minimising their proliferation.

Once 100% confluence was reached, the monolayers were scraped using a sterile p1000 pipette tip along the middle to create a straight-line “scratch”. After cell debris was washed off, the wells were replenished with fresh incomplete-LSGS medium.

The plates were then exposed to normal air, 75% argon or 75% nitrogen for 2 hours. From 0 to 16 hours post-exposure, wells were examined every 4 hours. At 16 hours, when the scratch of argon group appeared to have been completely closed by migrating cells, the assay was terminated. The cells were then fixed with 4% paraformaldehyde (Fischer, UK) for 8 minutes and stained with 0.1% crystal violet (Sigma-Aldrich, UK) for 10 minutes. Images were taken as described in the previous section.

### Western Blot Analysis

HUVEC cell sample was stored in a −80° C. freezer until use. Cell samples were homogenized in ice-cold cell lysis buffer (20 mM Tris pH 7.5, 1% Triton X-100, 150 mM sodium chloride, 1% pyrophosphate, 1% betaglycophosphate, 1% NP-40, 0.1% SDS, 1 mM EDTA, 100 μg/ml phenylmethylsulfonyl fluoride, 1 μg/ml aprotinin). All homogenates were then centrifuged for 20 minutes at 15,000 rpm at 4° C. The supernatants were then extracted and protein quantification was carried out using a Bradford protein quantification assay kit (Bio-Rad, Hemel Hempstead, UK). Protein extracts (10 μg) from each sample were denatured in NuPAGE LDS sample buffer (Invitrogen, Paisley UK) using a thermomixer at 70° C. for 10 minutes. The proteins were then separated using sodium polyacrylamide gel electrophoresis (NuPAGE, Invitrogen, Paisely, UK) before being transferred to a polyvinylidene fluoride membrane (Invitrogen, Paisely, UK).

Membranes were then blocked in 5% non-fat powdered milk in TBST (50 mM Tris-HCl, 150 mM sodium chloride, 0.1% Tween 20, pH 7.4). Membranes were then incubated overnight with either angiopoietin 1 (1:1000) (Santa Cruz, USA) or rabbit TGF-β (1:1000), VEGF, α-SMA (Sigma-Aldrich, Missouri, USA) with TBST at 4° C. This was followed by 2 hour incubation at room temperature with a secondary antibody: Donkey anti-rabbit HRP-conjugated antibody (1:1000) (Cell Signaling, Massachusetts, USA). Membranes were then treated with the ECL chemoluminescence system (Amersham Biosciences, Little Chalfont, UK) and then images were developed using the Syngene G:Box gel analysis system (Syngene, Cambridge, UK). Protein bands were then analyzed by densitometric quantification with Adobe PhotoShop (Adobe Systems, California, USA). Membranes were then reprobed with monoclonal anti-α-tubulin antibody. This allowed for normalization of the relative expression levels of the primary protein of interest between groups on the same membrane by means of calculation of the ratio of the protein of interest to β-actin.

### Outcomes

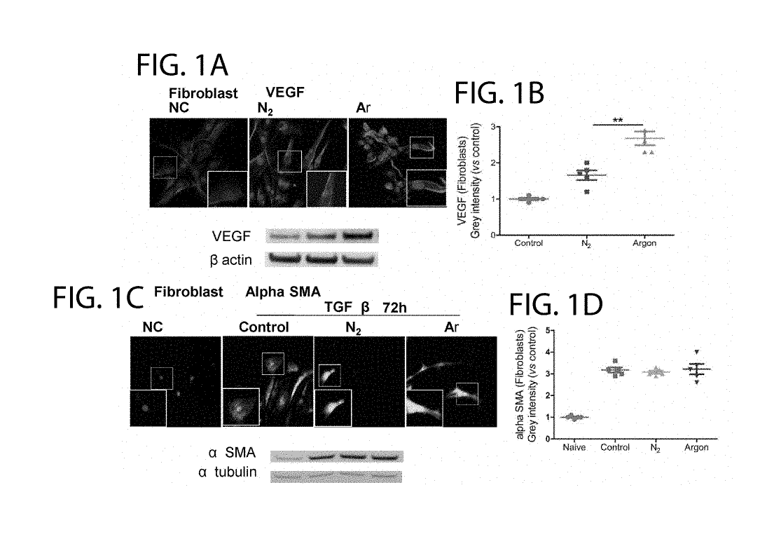

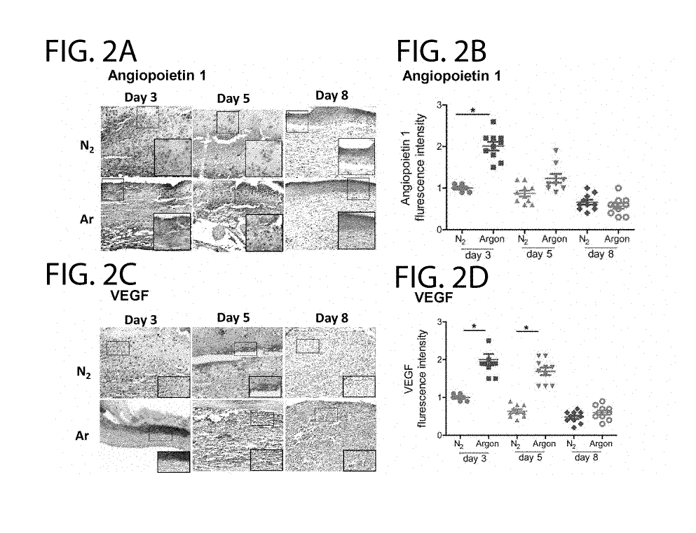

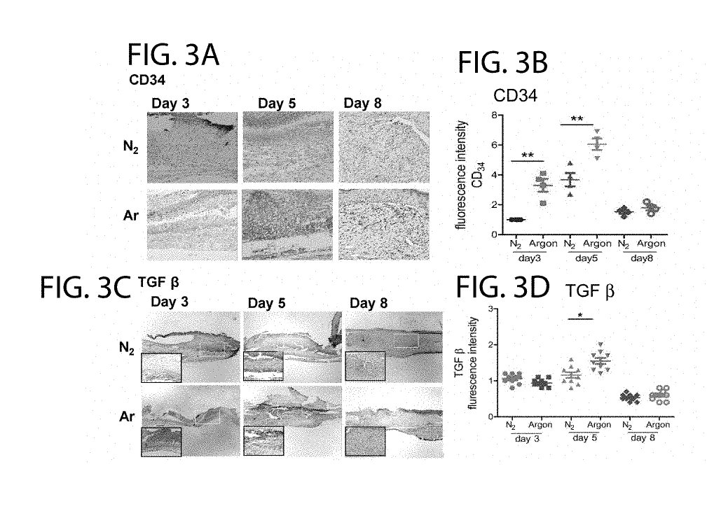

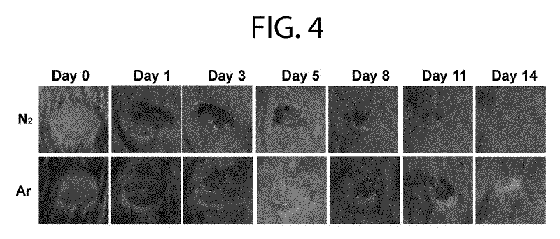

Exposure of argon enhanced VEGF and a-SMA expression in vitro (FIG. 1). Argon enhanced Angiopoietin 1 and vascular endothelial growth factor (VEGF) expression in vivo (FIG. 2) and enhanced CD34 and TGF-β in vivo (FIG. 3). Skin wound healing in mice was accelerated by treatment with Argon (FIG. 4).

FIG. 1 Argon Enhanced VEGF and a-SMA Expression In Vitro.

Fibroblasts were incubated in DMEM/low glucose media containing 10% FBS, saturated with N₂ (75% N₂+25% O₂) or Argon (75% Argon+25% O₂). (A) VEGF expression were detected by immunoflurance staining, VEGF expression (red), nuclei were counterstained with DAPI (blue) in fibroblasts 24 h after treatment; VEGF expression in fibroblasts were detected by western blotting; right lane: the intensity of VEGF was quantified by densitometry and normalized by 21% O₂ (mean±S.D, n=5). ** p<0.01. Fibroblasts were incubated in DMEM/low glucose media containing 10% FBS, saturated with N₂ (75% N₂+25% O₂) or Argon (75% Argon+25% O₂), Fibroblasts from Control, N₂ and Argon were incubated in culture media with 10 ng/ml TGF (3. (B) a SMA expression were detected by immunoflurance staining, a SMA expression (green), nuclei were counterstained with DAPI (blue) in fibroblasts 72 h after treatment; left lane: a SMA expression in fibroblasts were detected by western blotting; right lane: the intensity of a SMA was quantified by densitometry and normalized by 21% O₂ (mean±S.D, n=6). ** p<0.01 versus Control.

FIG. 2 Argon Enhanced Angiopoietin 1 and Vascular Endothelial Growth Factor (VEGF) Expression In Vivo

(A) Angiopoietin 1 staining of adult mouse cutaneous wounds. Representative images were shown at 3, 5, 8 day after wound. Right lane: the intensity of Angiopoietin 1 was quantified by densitometry and normalized by 75% N₂ at 3 day (mean±S.D, n=10), * p<0.05. (B) VEGF staining of adult mouse cutaneous wounds. Representative images were shown at 3, 5, 8 day after wound. The intensity of VEGF was quantified by densitometry and normalized by 75% N₂ at 3 day (mean±S.D, n=10), * p<0.05.

FIG. 3 Argon Enhanced CD34 and TGF-β In Vivo

(A) Immunohistochemical analyses using anti-CD34 in skin wound samples from the mice treated with Nitrogen and Argon. CD34 was used to mark the new vessels in the wound area. The numbers of new vessels per a high-power microscopic field (×100) in 10 fields/a slide was counted. All values represent the mean±S.D (n=12 mice). ** p<0.01. (B) TGFβ staining of adult mouse cutaneous wounds. Representative images were shown at 3, 5, 8 day after wound. The intensity of TGFβ was quantified by densitometry and normalized by 75% N₂ at 3 day (mean±S.D, n=10), * p<0.05.

FIG. 4 Skin Wound Healing in Mice Treated with 75% Nitrogen and 75% Argon.

A Macroscopic changes in skin wound site in a mice treated with 75% nitrogen or Argon. Day 0 picture was taken immediately after injury. Representative images from 2 individual animals in both groups are shown.

### REFERENCES

1. Velnar, T., T. Bailey, and V. Smrkolj, The wound healing process: an overview of the cellular and molecular mechanisms. J Int Med Res, 2009. 37(5): p. 1528-42.
2. Lamalice, L., F. Le Boeuf, and J. Huot, Endothelial cell migration during angiogenesis. Circ Res, 2007. 100(6): p. 782-94.
3. Falanga, V., Wound healing and its impairment in the diabetic foot. Lancet, 2005. 366(9498): p. 1736-43.
4. Game, F. L., et al., A systematic review of interventions to enhance the healing of chronic ulcers of the foot in diabetes. Diabetes Metab Res Rev, 2012. 28 Suppl 1: p. 119-41.
5. Jawad, N., et al., Neuroprotection (and lack of neuroprotection) afforded by a series of noble gases in an in vitro model of neuronal injury. Neurosci Lett, 2009. 460(3): p. 232-6.
6. Koblin, D. D., et al., Minimum alveolar concentrations of noble gases, nitrogen, and sulfur hexafluoride in rats: helium and neon as nonimmobilizers (nonanesthetics). Anesth Analg, 1998. 87(2): p. 419-24.

1. A method of inducing factors that stimulate hemangiogenic cells, said method comprising of contacting a noble gas with a mammalian tissue at sufficient concentration and duration to stimulate production of said factor.

2. The method of claim 1, wherein, said hemangiogenic cell is a cell capable of hematopoiesis.

3. The method of claim 1, wherein said hemangioblast cells is an endothelial progenitor cell.

4. The method of claim 3, wherein said endothelial progenitor cell is capable of generating endothelial cells in tissue culture.

5. The method of claim 1, wherein said noble gas is selected from a group comprising of: a) helium; b)

6. The method of claim 1, wherein said mammalian tissue contacted with said noble gas comprises of endothelial cells.

7. The method of claim 1, wherein said mammalian tissue contacted with said noble comprises of myeloid lineage cells.

8. The method of claim 1, wherein said stimulation of hemangiogenic cells is induction of proliferation.

9. The method of claim 1, wherein said stimulation of hemangiogenic cells is induction of cytokine production.

10. The method of claim 1, wherein said stimulation of hemangiogenic cells is induction of receptors associated with chemotaxis.

11. The method of claim 1, wherein said factor capable of stimulating hemangiogenic cells is VEGF.

12. The method of claim 1, wherein said factor capable of stimulating hemangiogenic cells is angiopoietin.

13. A method of inducing production of VEGF in a fibroblast comprising of contacting said fibroblast with a noble gas for a sufficient concentration and exposure time to induce production of said VEGF.

14. The method of claim 13, wherein said noble gas is argon.

15. The method of claim 14, wherein said noble gas is argon at a concentration of 75% argon with 25% oxygen.

16. A method of stimulating production of an angiogenic factor in an injured tissue comprising administering a therapeutically sufficient concentration of a noble gas to said injured tissue.

17. The method of claim 16, wherein said noble gas is argon.

18. The method of claim 16, wherein said argon is administered at a concentration of 75% argon and 25% oxygen.

19. The method of claim 16, wherein said angiogenic factor is angiopoietin.

20. The method of claim 16, wherein said angiogenic factor is VEGF.
